(19) United States
(12) Patent Application Publication
(10) Pub. No.: US 2017/0000433 A1
(43) Pub. Date: Jan. 5, 2017

(54) BREAST IMAGING APPARATUS, METHOD OF CONTROLLING BREAST IMAGING APPARATUS, AND STORAGE MEDIUM

(71) Applicant: CANON KABUSHIKI KAISHA, Tokyo (JP)

(72) Inventors: Osamu Tsujii, Kawasaki-shi (JP); Tetsuo Shimada, Hachioji-shi (JP); Sakiko Yamaguchi, Tokyo (JP); Takahiro Noguchi, Tokyo (JP); Hitomi Ogasawara, Kawasaki-shi (JP); Chifuyu Inagaki, Tokyo (JP)

(21) Appl. No.: 15/184,283

(22) Filed: Jun. 16, 2016

(30) Foreign Application Priority Data
Jun. 30, 2015 (JP) 2015-131838

(51) Int. Cl. A61B 6/00; A61B 6/03

(52) U.S. Cl. CPC A61B 6/4417; A61B 6/502; A61B 6/032; A61B 6/54; A61B 6/4085; A61B 6/4208; A61B 6/4435; A61B 6/4476

(57) ABSTRACT

A breast imaging apparatus includes a radiation imaging unit capable of performing mammogram imaging and CT imaging. The breast imaging apparatus includes: a cover detection unit configured to detect a state of a cover that separates an object from the radiation imaging unit; and a control unit configured to switch between the mammogram imaging and the CT imaging based on the state.

### BACKGROUND OF THE INVENTION

Field of the Invention

The present invention relates to a breast imaging apparatus that performs mammography using radiation, a method of controlling the breast imaging apparatus, and a storage medium.

Description of the Related Art

Japanese Patent Laid-Open No. 2013-538668 discloses, as a breast imaging apparatus, an arrangement having a function of performing CBCT (Cone-Beam CT) imaging of a breast while rotating a radiation generation unit and a radiation detection unit by a rotation unit and a function of performing mammogram imaging while fixing the breast by a fixing unit.

Japanese Patent Laid-Open No. 2013-538668 discloses an arrangement for performing CBCT imaging of a breast of an object in a standing position and an arrangement for performing mammogram imaging. However, since there is no cover between the object and the apparatus, it may be difficult to ensure safety for the object when performing various kinds of imaging.

The present invention provides a breast imaging technique capable of performing both mammogram imaging and CT imaging while ensuring safety for an object when performing imaging.

### SUMMARY OF THE INVENTION

According to one aspect of the present invention, there is provided a breast imaging apparatus including a radiation imaging unit capable of performing mammogram imaging and CT imaging, comprising: a cover detection unit configured to detect a state of a cover that separates an object from the radiation imaging unit; and a control unit configured to switch between the mammogram imaging and the CT imaging based on the state.

According to another aspect of the present invention, there is provided a method of controlling of a breast imaging apparatus including a radiation imaging unit capable of performing mammogram imaging and CT imaging, comprising: detecting a state of a cover that separates an object from the radiation imaging unit; and switching between the mammogram imaging and the CT imaging based on the state.

According to the present invention, it is possible to provide a breast imaging technique capable of performing both mammogram imaging and CT imaging while ensuring safety for an object when performing imaging.

Further features of the present invention will become apparent from the following description of exemplary embodiments (with reference to the attached drawings).

### BRIEF DESCRIPTION OF THE DRAWINGS

FIG. 1 is a view showing the outer appearance of a breast imaging apparatus according to an embodiment at the time of mammogram imaging;

FIG. 2 is a view showing the outer appearance of the breast imaging apparatus according to the embodiment at the time of mammogram imaging;

FIG. 3 is a view showing the outer appearance of the breast imaging apparatus according to the embodiment at the time of CBCT imaging;

FIG. 4 is a view showing the outer appearance of the breast imaging apparatus according to the embodiment at the time of CBCT imaging;

FIGS. 5A and 5B are views for explaining the imaging operation of the breast imaging apparatus according to the embodiment;

FIG. 6 is a view for explaining processing of the control unit of the breast imaging apparatus according to the embodiment;

FIG. 7 is a view for explaining processing of the control unit of the breast imaging apparatus according to the embodiment;

FIGS. 8A and 8B are views showing the outer appearance of a breast imaging apparatus according to the second embodiment at the time of CBCT imaging;

FIGS. 9A and 9B are views showing the outer appearance of the breast imaging apparatus according to the second embodiment at the time of mammogram imaging;

FIGS. 10A to 10C are views showing states of front covers having an opening/closing structure;

FIG. 11 is a view for explaining processing of the control unit of the breast imaging apparatus according to the second embodiment; and

FIG. 12 is a view for explaining processing of the control unit of the breast imaging apparatus according to the second embodiment.

### DESCRIPTION OF THE EMBODIMENTS

Preferred embodiments of the present invention will now be described in detail with reference to the accompanying drawings. Note that the constituent elements described in the embodiments are merely examples. The technical scope of the present invention is determined by the scope of claims and is not limited by the following individual embodiments.

### First Embodiment

A breast imaging apparatus according to the embodiment is a breast imaging apparatus including a radiation imaging unit capable of performing mammogram imaging and CT imaging. The breast imaging apparatus includes a cover detection unit 152 that detects the state of a cover that separates an object from the radiation imaging unit, and a control unit 200 that switches between mammogram imaging and CT imaging based on the state of the cover. The cover detection unit 152 can detect the presence/absence of mounting of the cover or the position of the cover as a mounted state of the cover. That is, the cover detection unit 152 includes a first detection unit that detects the presence/absence of mounting of the cover as the mounted state of the cover, and a second detection unit that detects the position of the cover. For example, by the first detection unit, the cover detection unit 152 can detect the presence/absence of mounting of the cover that separates the object from the radiation imaging unit as the mounted state of the cover. For example, if the cover has a structure (opening/closing structure) divided into left and right portions, the cover detection unit 152 can detect a position corresponding to the open/closed state of the cover by the second detection unit. Detection of the position of the cover will be described in detail in the second embodiment. If the cover is mounted based on the detection result of the cover detection unit 152, the control unit 200 control CT imaging. If the cover is not mounted, the control unit 200 control mammogram imaging. A radiation imaging unit 2 includes a radiation generation unit 10 that generates radiation, a radiation detection unit 20 that detects the radiation, and a rotation unit 56 capable of rotating the radiation generation unit 10 and the radiation detection unit 20 in a state in which they face each other. In the following description, the cover that separates the object from the radiation imaging unit will be explained as a front cover 9.

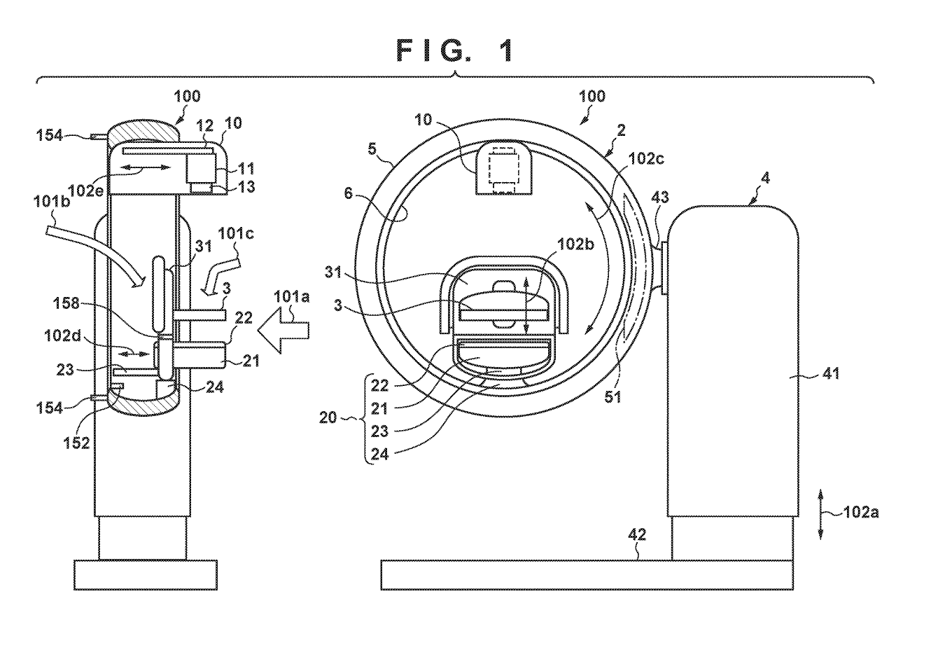

FIG. 1 is a view showing the outer appearance of a breast imaging apparatus 100 according to the first embodiment. In the first embodiment, an explanation will be made using the breast imaging apparatus 100 capable of performing mammogram imaging and CBCT (Cone-Beam CT) imaging as a radiation imaging apparatus. The radiation imaging unit 2 of the breast imaging apparatus 100 according to this embodiment includes the radiation generation unit 10 that includes a radiation tube 11 (for example, an X-ray tube) serving as a radiation source and generates radiation, the radiation detection unit 20 that includes a radiation detector 21 such as an FPD (Flat Panel Detector) and detects the radiation irradiation from the radiation generation unit 10, and the rotation unit 56 capable of rotating the radiation generation unit 10 and the radiation detection unit 20 in a state in which they face each other.

The rotation unit 56 of the radiation imaging unit 2 includes a ring-shaped rotary frame 6 in which, for example, the radiation generation unit 10 and the radiation detection unit 20 are arranged while facing each other, and a fixed frame 5 that rotatably holds the rotary frame 6 by a rotation sliding member (for example, a bearing).

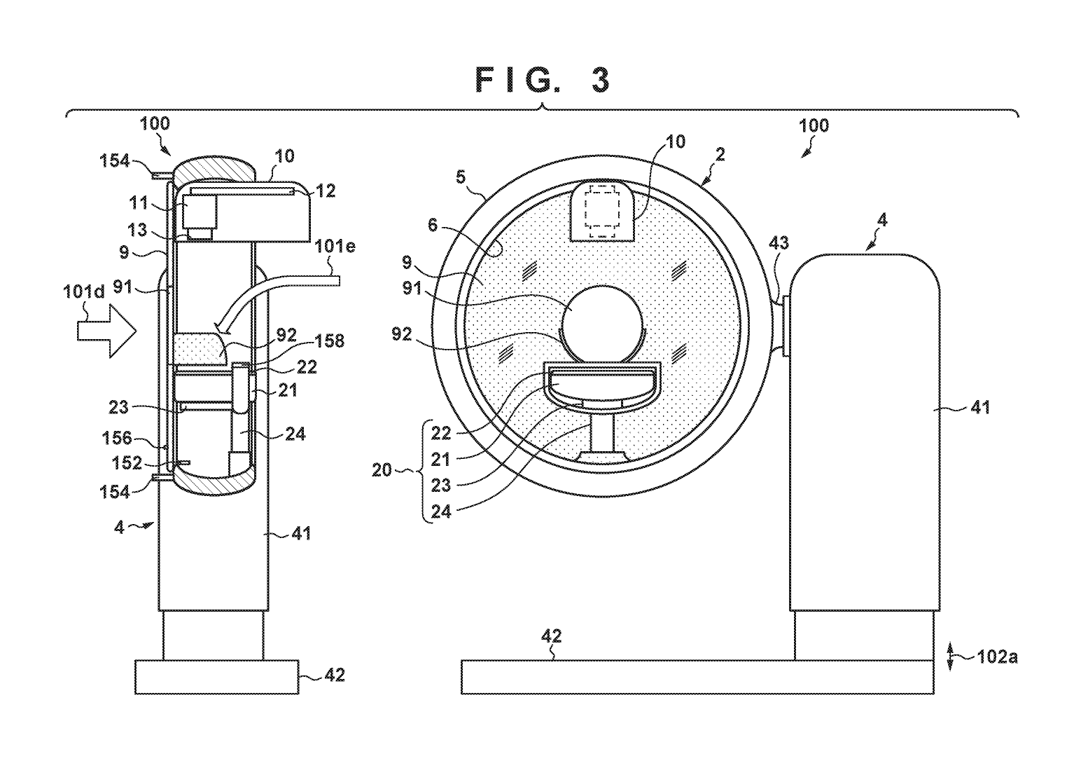

The radiation imaging unit 2 of the breast imaging apparatus 100 is configured to cause a breast that is a body part to be imaged to enter between a pressing plate 3 and the radiation detection unit 20 from the first side (the side of an arrow 101a) of the surface of revolution of the rotary frame 6 for mammogram imaging. The radiation imaging unit 2 of the breast imaging apparatus 100 is also configured to cause a breast that is a body part to be imaged to enter between the radiation generation unit 10 and the radiation detection unit 20 from the second side (an arrow 101d in FIG. 3) opposite to the first side of the surface of revolution of the rotary frame 6 for CT imaging (CBCT imaging in this embodiment).

That is, the radiation imaging unit 2 can implement a mode to perform imaging (mammogram imaging) in a state in which the body part of an object to be imaged is made to enter from the first side in the breast imaging apparatus 100 and sandwiched between the pressing plate 3 and the radiation detection unit 20 and a mode to perform imaging (CBCT imaging) while rotating the radiation generation unit 10 and the radiation detection unit 20 by the rotation unit 56 in a state in which the body part of an object to be imaged is made to enter between the radiation generation unit 10 and the radiation detection unit 20 from the second side opposite to the first side in the breast imaging apparatus 100.

FIG. 1 shows a state in which the breast imaging apparatus 100 captures a CC (Caranio Caudal) view of a mammogram. The rotation position of the rotary frame 6 is decided such that the radiation tube 11, the pressing plate 3, and the radiation detector 21 (radiation detection unit 20) are arranged in the vertical direction. A pressing plate support unit 31 supports the pressing plate 3, and can move the pressing plate 3 in a predetermined direction 102b (for example, a direction in which the pressing plate support unit 31 mounted on the rotary frame 6 moves upward to the rotation center of the rotary frame 6 or a direction in which the pressing plate support unit 31 moves downward to the rotary frame 6). The pressing plate support unit 31 is placed to be removable from the rotary frame 6. Note that the pressing plate support unit 31 may be placed to be removable from a constituent element integrated with the rotary frame 6, for example, the radiation detection unit 20, a detector moving unit 23, or an elevating unit 24. An imaging technician can remove the pressing plate support unit 31 together with the pressing plate 3. The imaging technician can adjust the distance between the pressing plate 3 and the radiation detection unit 20 by moving the pressing plate 3 by the pressing plate support unit 31. The breast of the object can be pressed by moving the pressing plate 3. In mammogram imaging, the breast arranged between the pressing plate 3 and the radiation detection unit 20 is pressed between the pressing plate 3 and the radiation detection unit 20 and undergoes radiation imaging.

The fixed frame 5 of the radiation imaging unit 2 is supported by a housing support unit 41 of a housing unit 4 via a fixed shaft 43. The housing support unit 41 is configured to be able to move vertically with respect to a housing fixing unit 42. The radiation imaging unit 2 is thus supported to be movable in the vertical direction (arrow 102a) with respect to the housing fixing unit 42.

A rotation motor 51 is attached to the distal end of the fixed shaft 43 that connects the housing unit 4 and the radiation imaging unit 2. The rotary frame 6 is rotatably connected to the rotation motor 51 via a bearing. The fixed frame 5 is stationarily connected to the fixed shaft 43. The rotary frame 6 is arranged inside the fixed frame 5. The bearing is arranged in the gap between the fixed frame 5 and the rotary frame 6. By driving the rotation motor 51, the rotary frame 6 can be rotated by 360° or more in a direction indicated by an arrow 102c with respect to the fixed frame 5.

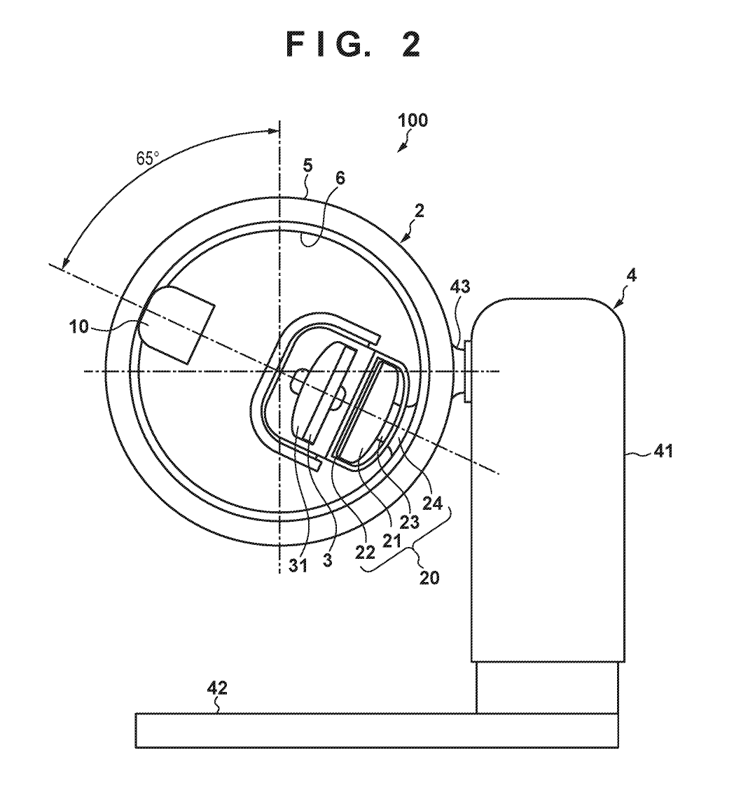

When capturing a CC view of a mammogram, the radiation tube 11, the radiation detector 21, and the pressing plate 3 are arranged in the vertical direction, as shown in FIG. 1. On the other hand, when capturing an MLO (Mediolateral Oblique) view of a mammogram by the breast imaging apparatus 100, the rotary frame 6 is rotated by a predetermined angle (for example, about) 65°) from the state shown in FIG. 1 and stopped, as shown in FIG. 2. Note that the stop state of the rotary frame 6 may be maintained by servo control or a brake. The breast that is the body part to be imaged is pressed between the radiation detector 21 and the pressing plate 3 and undergoes radiation imaging. By capturing such an MLO view, imaging of an armpit can be performed.

Referring back to FIG. 1, in mammogram imaging, the imaging technician can access the breast of the object via the hollow portion of the rotary frame 6, as indicated by an arrow 101b, arrange the breast between the pressing plate 3 and the radiation detector 21 of the breast imaging apparatus 100, and adjust the pressing. On the first side that is the breast insertion side upon mammogram imaging, the radiation tube 11, the radiation detector 21, and the pressing plate 3 are fixed such that they project in a first direction with respect to the surface of revolution of the rotary frame 6. For this reason, the imaging technician can also access the breast of the object from a side (between the surface of revolution and the object) of the breast imaging apparatus 100, as indicated by an arrow 101c, and adjust the pressing. The arrangement of the breast imaging apparatus 100 at the time of mammogram imaging has been described above.

CBCT imaging by the breast imaging apparatus 100 will be described next. The radiation generation unit 10 includes a radiation source moving unit 12 that can move and arrange the radiation tube 11 in the rotation axis direction (arrow 102e) of the rotary frame 6 for mammogram imaging and CT imaging. The radiation source moving unit 12 includes, for example, a rail on which the radiation tube 11 slides, and the imaging technician can manually move the radiation tube 11. Alternatively, the radiation tube 11 may be moved in the direction of the arrow 102e by the driving force of a linear motor or the like.

The radiation detection unit 20 includes the detector moving unit 23 that can move and arrange the radiation detector 21 in the rotation axis direction (arrow 102d) of the rotary frame 6 for mammogram imaging and CT imaging. The detector moving unit 23 includes a rail on which the radiation detector 21 slides, and the imaging technician can move the radiation detector 21 in the direction of the arrow 102d. Alternatively, the radiation detector 21 may be moved in the direction of the arrow 102d by the driving force of a linear motor or the like. The radiation detection unit 20 also includes the elevating unit 24 that moves the radiation detector 21 in the rotation center direction (arrow 102b) of the rotary frame 6 for mammogram imaging and CT imaging.

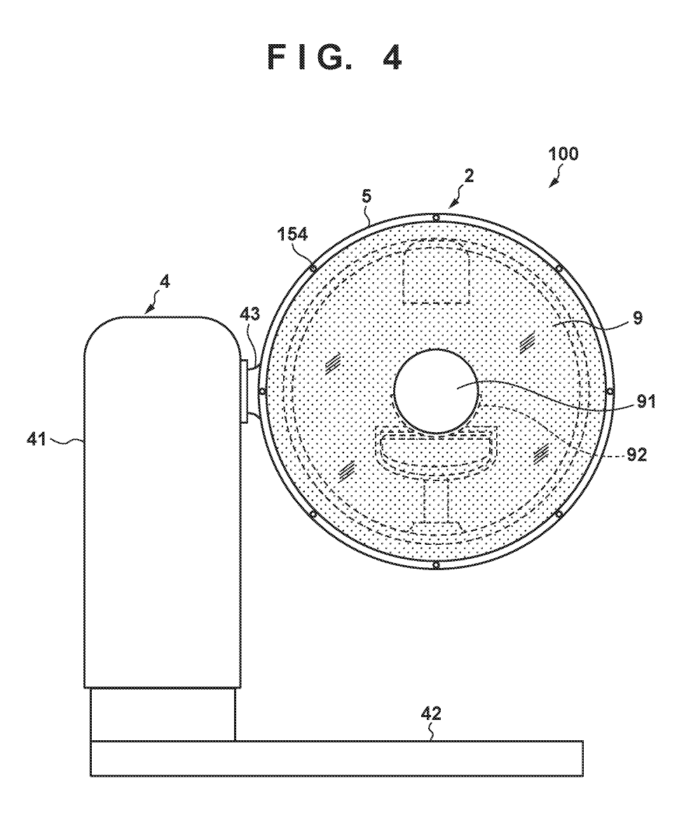

FIG. 3 shows a state in which the breast imaging apparatus 100 according to this embodiment performs CBCT imaging of a breast of an object. FIG. 4 is a view showing the outer appearance of the breast imaging apparatus 100 from the direction of the arrow 101d that is the insertion direction of the breast of the object. At the time of CBCT imaging, the breast is inserted from the second side opposite to the breast insertion side (first side) at the time of mammogram imaging (arrow 101d). In addition, the radiation tube 11 and the radiation detector 21 are moved to the second side opposite to the first side and arranged by the radiation source moving unit 12 and the detector moving unit 23. The radiation source moving unit 12 and the detector moving unit 23 may be configured to move the radiation tube 11 and the radiation detector 21 by motor driving or the like or manually. The radiation source moving unit 12 and the detector moving unit 23 can arrange the radiation tube 11 and the radiation detector 21 at positions where mammogram imaging can be executed for a breast inserted from the first side, and CBCT imaging can be executed for a breast inserted from the second side.

The pressing plate support unit 31 and the pressing plate 3 are removable from the rotary frame 6, that is, the radiation imaging unit 2. If the pressing plate support unit 31 and the pressing plate 3 are kept placed on the rotary frame 6, they hinder the imaging technician from accessing the breast of the object when performing CBCT imaging. Hence, at the time of CBCT imaging, the pressing plate support unit 31 is removed from the rotary frame 6 together with the pressing plate 3, as shown in FIGS. 3 and 4.

The elevating unit 24 of the radiation detection unit 20 moves the radiation detector 21 toward the rotation center of the rotary frame 6, thereby changing the distance between the radiation detector 21 and the radiation generation unit 10 (radiation tube 11). The radiation tube 11 and the radiation detector 21 are thus arranged in a positional relationship appropriate for CBCT imaging. On the second side of the radiation imaging unit 2 (fixed frame 5), the front cover 9 that separates the object from the radiation imaging unit is configured to be removable from the breast imaging apparatus. The front cover 9 has a function of preventing the object from interfering with the radiation detector 21 and the like when the rotary frame 6 rotates in CBCT imaging. This can ensure safety for the object when performing imaging. The front cover 9 that separates the object from the radiation imaging unit is circular, and is placed to be removable from the circular fixed frame 5. Note that the front cover 9 need only be fixed to a member immovable with respect to the rotation of the rotary frame 6, and may be placed on, for example, the fixed shaft 43.

The front cover 9 that separates the object from the radiation imaging unit is provided with an opening 91 to insert the body part of the object to be imaged. More specifically, the circular opening 91 used to make the breast of the object enter is provided at the center of the front cover 9. The front cover 9 includes, around the opening 91, a breast support 92 used to support the breast that has entered from the opening 91. Note that in this embodiment, the breast support 92 is fixed to the front cover 9. However, the present invention is not limited to this. For example, the breast support 92 may be fixed to the fixed frame 5 via a support member.

During CBCT imaging, radiation images are captured while rotating the rotary frame 6 with respect to the fixed frame 5, and a reconstruction unit (not shown) obtains a 3D reconstructed image. The front cover 9 fixed to the fixed frame 5 separates the object (not shown) from the radiation generation unit 10 and the radiation detection unit 20 which rotate during CBCT imaging. The breast of the object is held on the breast support 92 and therefore fixed during CBCT imaging.

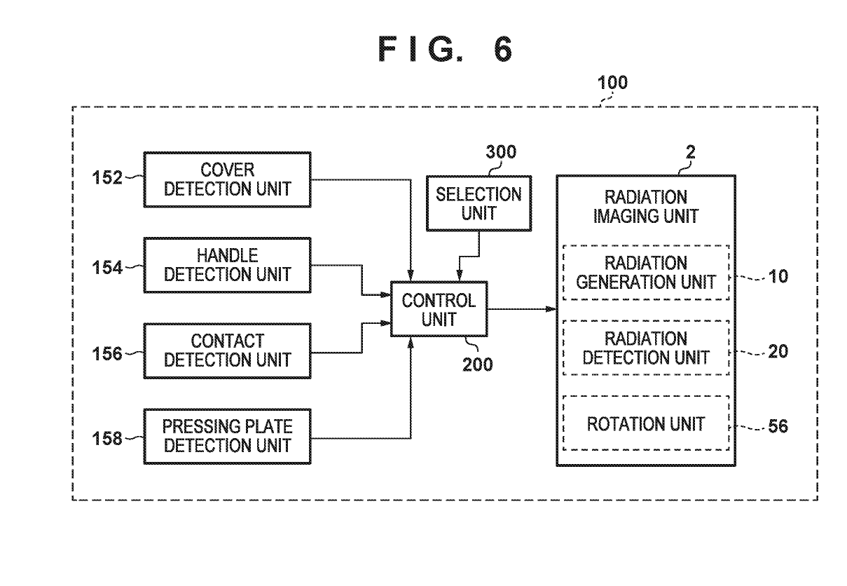

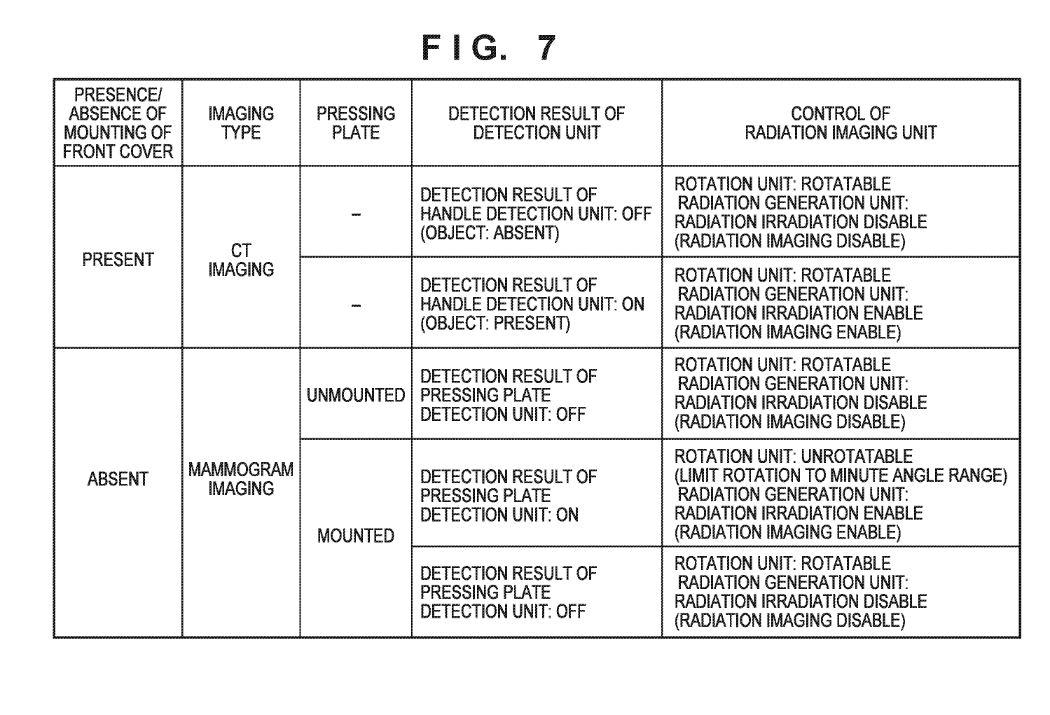

The breast imaging apparatus 100 according to this embodiment includes the cover detection unit 152 that detects the presence/absence of mounting of the front cover 9 that is arranged on the fixed frame 5 and separates the object from the radiation imaging unit. When the front cover 9 is mounted on the fixed frame 5, the cover detection unit 152 detects the mounting of the front cover 9, and changes to an ON state. The cover detection unit 152 outputs detection information representing the ON state to the control unit 200. On the other hand, when the front cover 9 is removed from the fixed frame 5, the cover detection unit 152 detects an unmounted state in which the front cover 9 has been removed, and changes to an OFF state. The cover detection unit 152 outputs detection information representing the OFF state to the control unit 200. The control unit 200 switches between mammogram imaging and CT imaging based on the detection result of the cover detection unit. Detailed control of the control unit 200 will be described later with reference to the control block diagram of FIG. 6 and FIG. 7.

Note that in the above-described example, the breast support 92 is connected along the periphery of the opening of the front cover 9. However, the present invention is not limited to this. For example, the breast support 92 need only be held immovably with respect to the rotation of the rotary frame 6 during CBCT imaging, and may be connected to, for example, the fixed frame 5. However, when the breast support 92 is connected to the front cover 9, a support member used to connect the fixed frame 5 and the breast support 92 is unnecessary. Hence, the imaging technician can easily access the breast of the object from the direction of an arrow 101e. In FIGS. 3 and 4, the front cover 9 has a removable form. However, the front cover 9 may have an opening/closing structure without hindrance to mammogram imaging. Note that the arrangement of the front cover 9 using the opening/closing structure will be described later in the second embodiment.

The imaging technician accesses the breast of the object that has entered from the first side of the fixed frame 5 and the rotary frame 6 via the opening 91 of the front cover 9, as indicated by the arrow 101e, and places the breast of the object on the breast support 92. Note that the front cover 9 can be configured to be transparent on both the first side and the second side. The front cover 9 can also be configured to be opaque on the object side (the side of the arrow 101d: second side) and transparent on the imaging technician side (the side of the arrow 101a in FIG. 1: first side). The front cover 9 formed to be opaque on the object side can prevent the object from becoming frightened by viewing the movement of the radiation generation unit 10 or the radiation detection unit 20 through the front cover 9. In addition, the front cover 9 formed to be transparent on the imaging technician side allows the imaging technician to visually confirm the state of the object and easily access the breast of the object.

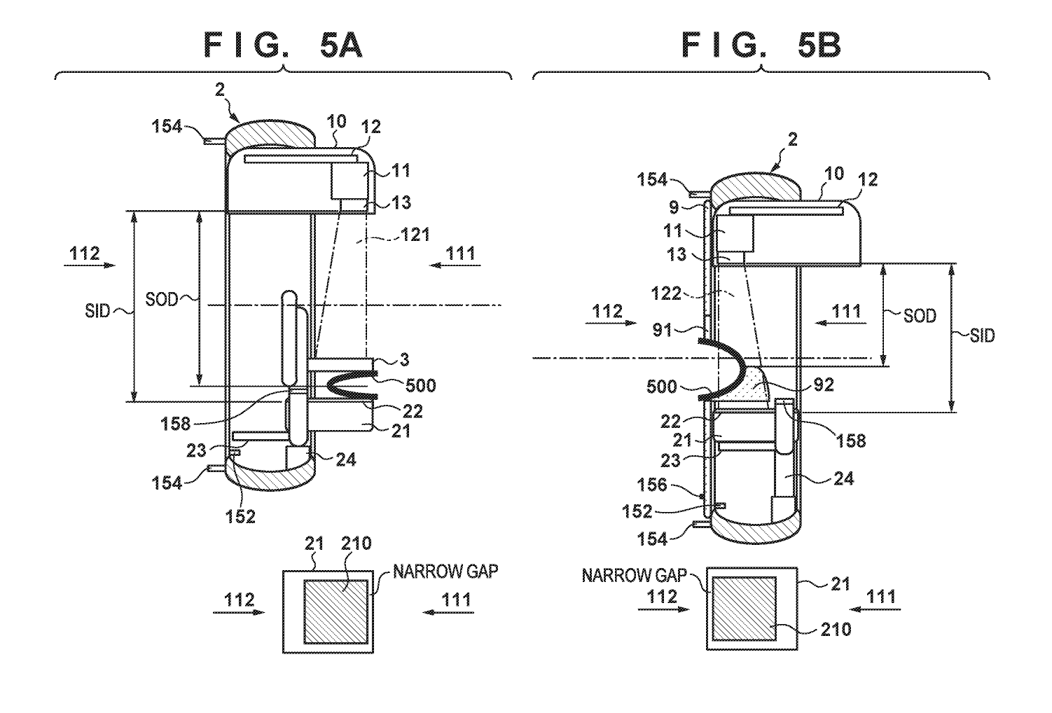

FIGS. 5A and 5B are views showing the mammogram imaging state and the CBCT imaging state of the breast imaging apparatus 100 according to this embodiment. As shown in FIGS. 5A and 5B, the access surface with respect to the rotary frame 6 for the object is reversed between mammogram imaging and CBCT imaging. The side of the access surface for the object at the time of mammogram imaging is defined as a first side 111, and the side of the access surface for the object in CBCT imaging is defined as a second side 112.

FIG. 5A is a side view in mammogram imaging. The object stands on the first side 111. In FIG. 5A, the rotary frame 6 is located at a position corresponding to CC imaging. In MLO imaging, the rotary frame 6 is rotated by about 65° (see FIG. 2). In the radiation generation unit 10, the radiation tube 11 is connected to the rotary frame 6 via the radiation source moving unit 12. The radiation detector 21, the pressing plate support unit 31, the pressing plate 3, and the like are connected to the rotary frame 6 via the elevating unit 24.

By the radiation generation unit 10 and the radiation detection unit 20 including these components, the radiation imaging unit 2 provides different imaging geometric systems in mammogram imaging and CT imaging. Different imaging geometric systems (SID (Source to Image Distance) and SOD (Source to Object Distance)) can thus be provided in mammogram imaging and CBCT imaging. In addition, since the radiation tube 11 and the radiation detector 21 project to the first side 111 with respect to the surface of revolution of the rotary frame 6 and the fixed frame 5, the imaging technician can access a breast 500 of the object from a side in mammogram imaging (the arrow 101c in FIG. 1). In addition, when the front cover 9 placed on the second side 112 is removed, the imaging technician can access the breast 500 of the object from the second side 112 via the hollow portion of the rotary frame 6 (the arrow 101b in FIG. 1).

Additionally, a radiation aperture 13 is placed in front of the radiation tube 11, and a grid 22 for scattered ray reduction is arranged in front of the radiation detector 21. Since the imaging geometric system changes between mammogram imaging and CBCT imaging, the radiation aperture 13 changes the aperture shape in accordance with mammogram imaging or CBCT imaging. Note that deformation of the opening shape of the radiation aperture 13 can be implemented by an arrangement that deforms the opening shape in accordance with a switching operation of the imaging technician or by exchanging the radiation aperture 13. In addition, the stripe direction, stripe frequency, and grid ratio of the grid 22 are also set in accordance with mammogram imaging or CBCT imaging. For example, the imaging technician exchanges the grid between mammogram imaging and CBCT imaging, thereby coping with each imaging mode.

FIG. 5B is a side view in CBCT imaging. In the breast imaging apparatus 100 according to this embodiment, the imaging technician can easily change the form of mammogram imaging shown in FIG. 5A to the form of CBCT imaging shown in FIG. 5B. That is, the imaging technician removes the pressing plate 3 (pressing plate support unit 31) from the breast imaging apparatus 100 shown in FIG. 5A, moves the radiation tube 11 to the second side 112, moves the radiation detector 21 to the second side 112, and moves the radiation detector 21 upward in FIG. 5A by the elevating unit 24. Note that an arrangement for moving the radiation detector 21 in the horizontal direction may be implemented by, for example, rotating the radiation detector about the elevating unit 24. However, if the radiation detector 21 that has rotated interferes with the rotary frame 6, the radiation detector 21 is configured to be rotatable, for example, after it is raised by the elevating unit 24 to the vicinity of the center of the rotary frame 6. To change the form of CBCT imaging shown in FIG. 5B to the form of mammogram imaging shown in FIG. 5A, for example, the imaging technician moves the radiation tube 11 and the radiation detector 21 to the first side 111, moves the radiation detector 21 downward in FIG. 5B by the elevating unit 24, and mounts the pressing plate 3 (pressing plate support unit 31) on the radiation detection unit 20.

In CBCT imaging, the object stands on the second side 112. The breast 500 of the object can be aligned with the opening 91 by vertically moving the housing support unit 41. For example, the radiation imaging unit 2 is moved downward by a distance indicated by an arrow 131, thereby aligning the breast 500 of the object with the opening 91. As described above, the pressing plate support unit 31 and the pressing plate 3 have removable structures and are removed in CBCT imaging. In addition, since the access surface for the object changes between mammogram imaging and CBCT imaging, the radiation tube 11 is placed so as to rotate by 180° when moving from the first side 111 to the second side 112. A radiation beam is formed to reduce the blind area (area that is not imaged) of the chest wall portion of the object small, as indicated by radiation beam shapes 121 and 122 in FIGS. 5A and 5B. Since the radiation beam is asymmetrical, the radiation tube 11 needs to be rotated. Note that a radiation beam suitable for each imaging may be formed by the radiation aperture 13 without rotating the radiation tube 11. That is, the radiation generation unit 10 can rotate the radiation shape (the radial shape of radiation) from the radiation tube 11 serving as a radiation source by 180° about the radial direction from the rotation center of the rotary frame 6 between mammogram imaging and CBCT imaging.

The detector moving unit 23 can mount the radiation detector 21 in a state in which the radiation detector 21 is rotated by 180° about the radial direction from the rotation center of the rotary frame 6 between mammogram imaging and CBCT imaging. This is because the access surface for the object changes between mammogram imaging and CBCT imaging. For example, the radiation detector 21 for mammography has a narrow gap (the distance from the outer edge of the sensor to a detection area 210 is 5 mm or less) along only one side of the detection area 210 to reduce the blind area of the chest wall portion, as shown in FIGS. 5A and 5B. For this reason, the radiation detector 21 can be moved and placed so as to rotate by 180° such that the narrow gap side is directed to the object, as shown in FIGS. 5A and 5B. Note that in the arrangement that rotates the radiation detector 21 about the elevating unit 24 to move the radiation detector 21, the narrow gap side is directed to the object by the rotation.

The breast imaging apparatus 100 according to this embodiment also includes an object detection unit that detects the object in front of the cover. As a detailed example of the object detection unit, the breast imaging apparatus 100 includes a handle detection unit 154 that is arranged in front of the fixed frame 5 or the front cover 9 and detects handle grip by the object (FIGS. 5A and 5B shows a state in which the handle detection unit 154 is arranged on the fixed frame 5). A plurality of handle detection units 154 are arranged at a predetermined interval along the circumferential direction of the circular fixed frame 5 (front cover 9). The handle detection units 154 are arranged so as to project to the front side (second side (the arrow 101d in FIG. 3)) of the fixed frame 5 (front cover 9). For example, when the object standing in front of the front cover 9 reaches out the left and right hands upward or downward and grips the handles of the handle detection units 154, the handle detection units 154 detect the presence of the object standing in front of the front cover 9. In addition, the object can fix the posture in imaging by gripping the handles of the handle detection units 154.

When the plurality of handle detection units 154 are arranged, the object can grip the handles of the handle detection units 154 provided at positions easy to grip in accordance with the state of the body. For example, when the object grips the handles of the handle detection units 154 arranged on the upper side of the fixed frame 5 or the front cover 9, the object is fixed in a posture stretched upward, and the posture of the object is stabilized. On the other hand, an object who cannot raise the arms can fix the posture by gripping the handles of the handle detection units 154 arranged on the lower side of the fixed frame 5 or the front cover 9. If the breast of the object moves during imaging, an artifact may be generated in a 3D reconstructed image. However, generation of the artifact can be suppressed by fixing the posture of the object at the time of imaging by making it grip the handles.

When the object grips a handle, the handle detection unit 154 detects the gripped state of the handle by the object and changes to an ON state. The handle detection unit 154 outputs detection information representing the ON state to the control unit 200. On the other hand, when the object releases the handle, the handle detection unit 154 detects an ungripped state in which the object has taken the hand off the handle, and changes to an OFF state. The handle detection unit 154 outputs detection information representing the OFF state to the control unit 200. Detailed control of the control unit 200 will be described later with reference to the control block diagram of FIG. 6 and FIG. 7.

The arrangement of the object detection unit that detects the object in front of the cover is not limited to the arrangement of the above-described handle detection unit 154. For example, a detection unit that detects the presence/absence of an object may be arranged in front of the front cover 9. That is, as the object detection unit that detects the object in front of the cover, the breast imaging apparatus 100 may also arrange a contact detection unit 156 that is arranged in front of the front cover 9 and detects the presence/absence of the object. The contact detection unit 156 can be formed from a contact type sensor that detects the presence/absence of the object when the object standing in front of the front cover 9 comes into contact with the detection surface of the contact detection unit 156. For example, even if the object cannot grip the handles of the handle detection units 154 depending on the state of the body, the contact detection unit 156 can detect the presence/absence of the object.

In the control of CT imaging, the control unit 200 can control the operations of the radiation generation unit 10 and the rotation unit 56 based on the detection result of the cover detection unit 152 and the detection result of the object detection unit. Here, the object detection unit includes the handle detection unit 154 and the contact detection unit 156 described above.

When performing CBCT imaging, the pressing plate support unit 31 and the pressing plate 3 are removable from the rotary frame 6, that is, the radiation imaging unit 2 to allow the imaging technician to easily access the breast of the object. The breast imaging apparatus 100 according to this embodiment includes a pressing plate detection unit 158 that detects that the body part of the object to be imaged is pressed by the pressing plate 3 mounted on the radiation imaging unit 2.

The imaging technician can adjust the distance between the pressing plate 3 and the radiation detection unit 20 by moving the pressing plate 3. When the distance between the moved pressing plate 3 and the radiation detection unit 20 is equal to or less than a set threshold, the pressing plate detection unit 158 detects pressing of the breast by the pressing plate 3 and changes to an ON state (pressed state). The pressing plate detection unit 158 outputs detection information representing the ON state to the control unit 200. On the other hand, when the distance between the pressing plate 3 and the radiation detection unit 20 is more than the set threshold, the pressing plate detection unit 158 does not detect pressing of the breast by the pressing plate 3 and changes to an OFF state (unpressed state). The pressing plate detection unit 158 outputs detection information representing the OFF state to the control unit 200. Note that when the pressing plate 3 (pressing plate support unit 31) is removed from the radiation imaging unit 2, the pressing plate detection unit 158 does not detect the pressing of the breast by the pressing plate 3, and outputs detection information representing the OFF state to the control unit 200. In the control of mammogram imaging, the control unit 200 can control the operations of the radiation generation unit 10 and the rotation unit 56 based on the detection result of the cover detection unit 152 and the detection result of the pressing plate detection unit 158. Detailed control of the control unit 200 will be described later with reference to the control block diagram of FIG. 6 and FIG. 7.

FIG. 6 is a block diagram showing for explaining processing of the control unit 200 of the breast imaging apparatus 100. The breast imaging apparatus 100 includes the cover detection unit 152, the handle detection unit 154, the contact detection unit 156, and the pressing plate detection unit 158 as the components of the detection unit. Each detection unit is connected to the control unit 200, and the detection result of each detection unit is input to the control unit 200. The control unit 200 controls the overall operation of the breast imaging apparatus 100. More specifically, the control unit 200 can control the operation of the radiation imaging unit 2 (the radiation generation unit 10, the radiation detection unit 20, the rotation unit 56, and the like) of the breast imaging apparatus 100 based on the detection result of each detection unit. A selection unit 300 that selects an imaging type is connected to the control unit 200. The control unit 200 can switch between mammogram imaging and CT imaging based on the selected imaging type. The selection unit 300 can select the imaging type based on an input by the imaging technician via a user interface (display unit) (not shown). Note that the selection unit 300 has a communication function capable of performing communication (wired communication or wireless communication) with an external apparatus (external system). The selection unit 300 can obtain the imaging type from the external apparatus (external system) by communication. The control unit 200 can switch between mammogram imaging and CT imaging based on the imaging type obtained by not selection of the selection unit 300 but communication of the selection unit 300.

FIG. 7 is a view for explaining processing of the control unit 200 of the breast imaging apparatus 100. When detection information of an ON state representing the mounted state of the front cover 9 is input from the cover detection unit 152 to the control unit 200, the control unit 200 determines that the front cover 9 is mounted (front cover mounting: present) and controls the imaging type of the radiation imaging unit 2 to CT imaging (CBCT imaging).

On the other hand, when detection information of an OFF state representing the unmounted state of the front cover 9 is input from the cover detection unit 152 to the control unit 200, the control unit 200 determines that the front cover 9 is unmounted (front cover mounting: absent) and controls the imaging type of the radiation imaging unit 2 to mammogram imaging. That is, the control unit 200 performs imaging control to switch between mammogram imaging and CT imaging based on the detection result of the cover detection unit.

In the control of CT imaging (CBCT imaging), when detection information of an OFF state representing the ungripped state of a handle by the object is input from the handle detection unit 154 to the control unit 200, the control unit 200 controls the rotation unit 56 of the radiation imaging unit 2 to a rotatable state but controls the radiation generation unit 10 to a radiation irradiation disable state. In this case, the imaging operation of the radiation imaging unit 2 is disabled by the control of the control unit 200. Even if the detection information of the OFF state is input from the handle detection unit 154 to the control unit 200, the rotation unit 56 is controlled to the rotatable state because the rotary frame 6 needs to be rotated when returning to the initial position of CBCT imaging before shifting from mammogram imaging to CBCT imaging. Regardless of whether the detection result of the handle detection unit 154 indicates the ON state or OFF state, the control unit 200 controls the rotation unit 56 to the rotatable state. Hence, in the operation of the rotary frame 6 when returning to the initial position of CBCT imaging, the operability for the imaging technician can be improved.

In the control of CT imaging (CBCT imaging), when detection information of an ON state representing the gripped state of a handle by the object is input from the handle detection unit 154 to the control unit 200, the control unit 200 controls the rotation unit 56 of the radiation imaging unit 2 to a rotatable state, and controls the radiation generation unit 10 to a radiation irradiation enable state. In this case, the control unit 200 controls to enable imaging by the radiation imaging unit 2. That is, if the object does not grip the handle of the handle detection unit 154 (OFF state), CT imaging (CBCT imaging) cannot be started.

When the object releases the handle of the handle detection unit 154 during CT imaging (CBCT imaging), the handle detection unit 154 changes from the ON state to the OFF state. Detection information of the OFF state is then input from the handle detection unit 154 to the control unit 200. Based on the detection information of the handle detection unit 154 representing the OFF state, the control unit 200 controls the radiation generation unit 10 and stops radiation irradiation from the radiation generation unit 10. That is, the control unit 200 controls the radiation generation unit 10 of the radiation imaging unit 2 to a radiation irradiation disable state, and the imaging operation of the radiation imaging unit 2 is disabled by the control of the control unit 200.

Note that FIG. 7 shows the handle detection unit as an example of the detection result of the object detection unit that detects the object in front of the cover. However, the control unit 200 can also control the operation of the radiation imaging unit 2 using the detection result of not the handle detection unit but the contact detection unit 156 that detects the presence/absence of the object.

In the control of mammogram imaging, when detection information of an OFF state representing the unpressed state of the breast by the pressing plate 3 is input from the pressing plate detection unit 158 to the control unit 200, the control unit 200 controls the rotation unit 56 of the radiation imaging unit 2 to a rotatable state but controls the radiation generation unit 10 to a radiation irradiation disable state. In this case, the imaging operation of the radiation imaging unit 2 is disabled by the control of the control unit 200.

In the control of mammogram imaging, when detection information of an ON state representing the pressed state of the breast by the pressing plate 3 is input from the pressing plate detection unit 158 to the control unit 200, the control unit 200 controls the rotation unit 56 of the radiation imaging unit 2 to an unrotatable state (limits the rotation to a minute angle range (for example, 1° to 2°)), and controls the radiation generation unit 10 to a radiation irradiation enable state. In this case, the imaging operation of the radiation imaging unit 2 is enabled by the control of the control unit 200. The rotation is limited to the minute angle range (for example, 1° to 2°) because the angle of MLO may finely be adjusted in the breast pressed state. To cope with such fine adjustment, even if the pressing plate detection unit 158 outputs the detection information of the ON state representing the pressed state, the control unit 200 can also control the radiation generation unit 10 to the radiation irradiation enable state in a state in which the rotation angle of the rotation unit 56 (rotary frame 6) is limited to a minute angle (for example, 1° to 2°).

In addition, a method of controlling a breast imaging apparatus including a radiation imaging unit capable of performing mammogram imaging and CT imaging includes a cover detection step (step S1) of detecting the mounted state of a cover that separates an object from the radiation imaging unit, and a control step (step S2) of switching between mammogram imaging and CT imaging based on the mounted state.

### Second Embodiment

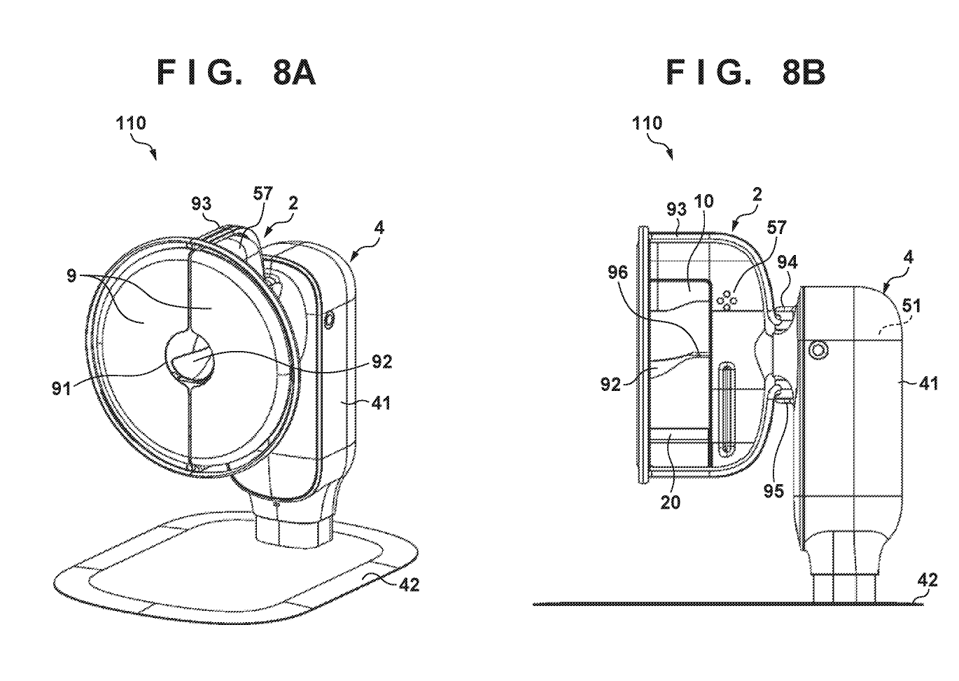

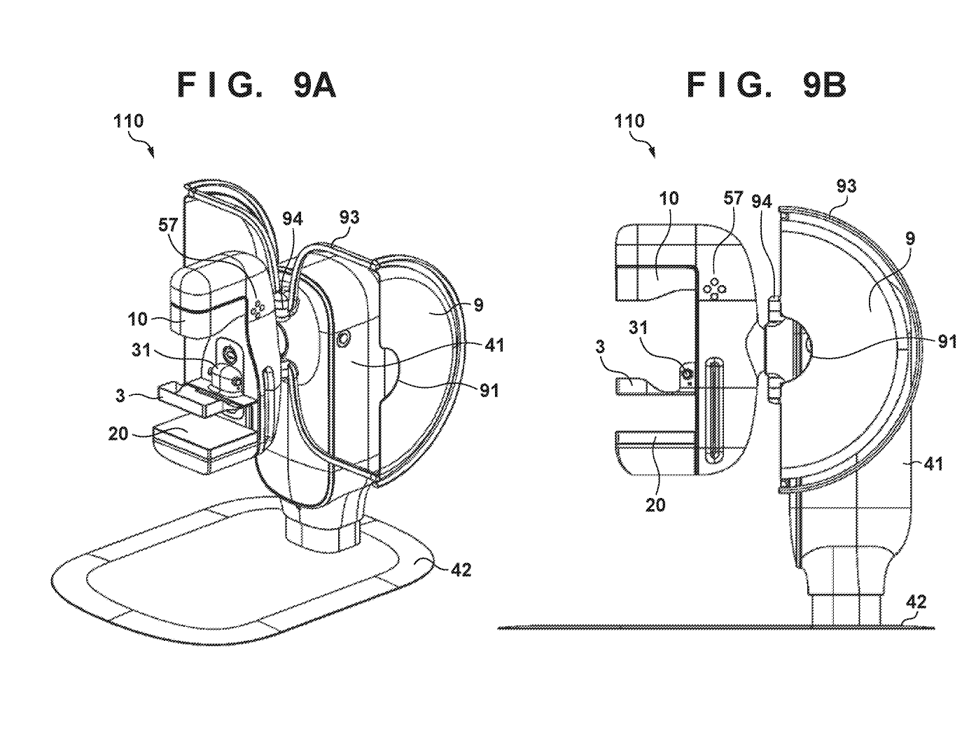

FIGS. 8A and 8B and FIGS. 9A and 9B are views showing the outer appearance of a breast imaging apparatus 110 according to the second embodiment. In the second embodiment, an explanation will be made using the breast imaging apparatus 110 capable of performing mammogram imaging and CBCT (Cone-Beam CT) imaging as a radiation imaging apparatus. In the first embodiment, an arrangement that performs mammogram imaging from the first side and CBCT imaging from the second side opposite to the first side has been described. In this embodiment, the arrangement of the breast imaging apparatus 110 capable of performing mammogram imaging and CBCT imaging on the same side will be described.

FIGS. 8A and 8B are views for explaining the schematic arrangement of the breast imaging apparatus 110 according to the second embodiment when performing CBCT imaging. FIG. 8A shows the apparatus in a state in which front covers 9 are mounted. FIG. 8B shows a state in which the apparatus with the front covers 9 being mounted is viewed from a side.

The same reference numerals as in the first embodiment denote the same components such as a radiation generation unit 10, a radiation detection unit 20, and a housing unit 4. To avoid a repetitive description, a description of the radiation generation unit 10, the radiation detection unit 20, the housing unit 4, and the like will be omitted.

A rotation unit 57 is connected to a rotation motor 51 arranged in the housing unit 4 and rotatably supported via a rotation support member such as a bearing. The rotation unit 57 functions like the rotary frame 6 in the first embodiment. The radiation generation unit 10 and the radiation detection unit 20 are connected to the rotation unit 57 in a state in which they face each other. The radiation imaging unit 2 of the breast imaging apparatus 110 according to this embodiment includes the radiation generation unit 10, the radiation detection unit 20 that detects radiation irradiation from the radiation generation unit 10, and the rotation unit 57 capable of rotating the radiation generation unit 10 and the radiation detection unit 20 in a state in which they face each other.

In the breast imaging apparatus 110 according to this embodiment, a breast support 92 is held by the housing unit 4 via a holding member 96 and held immovably with respect to the rotation of the rotation unit 57. Note that if the breast support 92 and the holding member 96 are kept held by the housing unit 4, they hinder an imaging technician from doing imaging when performing mammogram imaging. Hence, at the time of mammogram imaging, the breast support 92 is removed from the housing unit 4 together with the holding member 96, as shown in FIGS. 9A and 9B. In CBCT imaging, the breast support 92 is held by the housing unit 4 together with the holding member 96, as shown in FIGS. 8A and 8B.

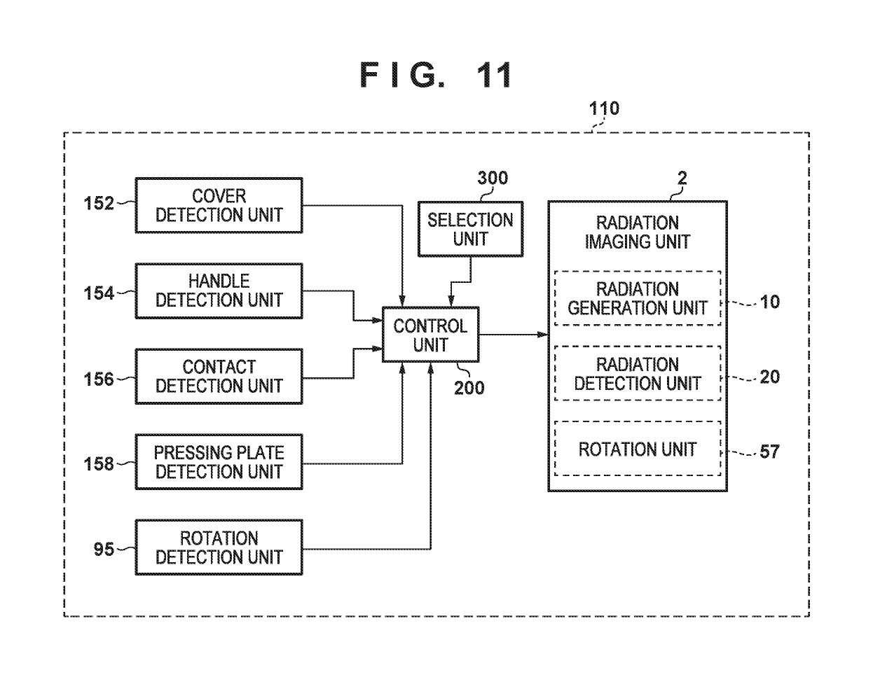

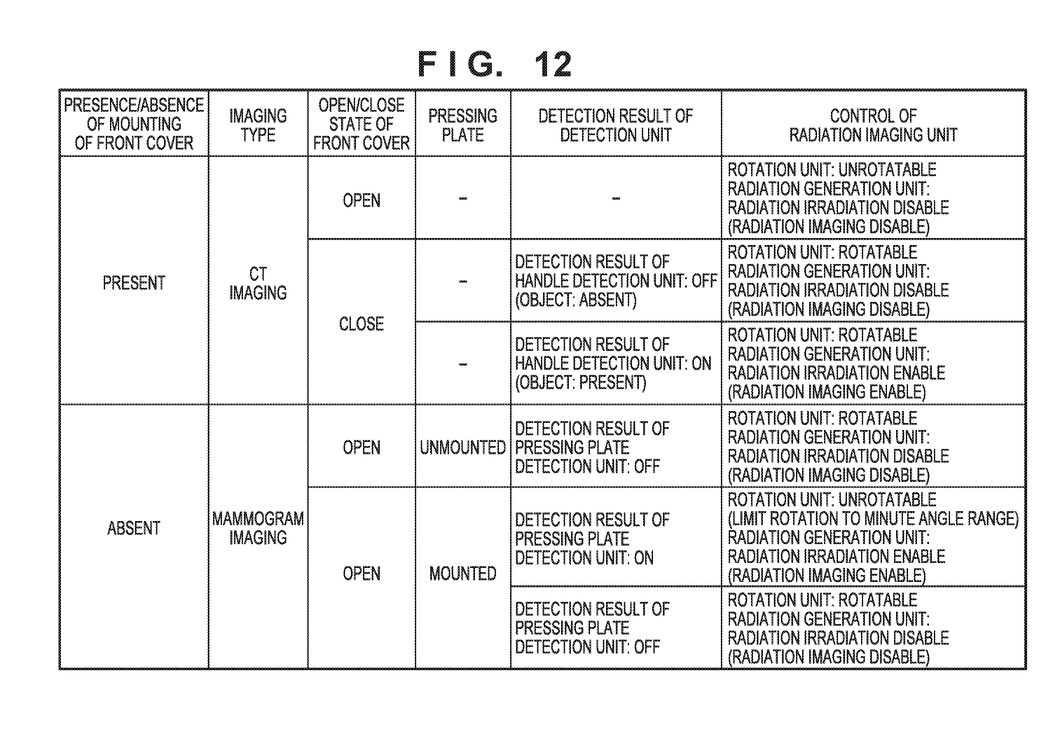

The breast imaging apparatus 110 according to this embodiment includes a rotation support unit 94 arranged in the housing unit of the breast imaging apparatus, a support member 93 rotatably supported with respect to the rotation axis of the rotation support unit 94 as the rotation center and configured to be able to mount a front cover, and a rotation detection unit 95 that detects the rotation of the rotation axis. A control unit 200 can determine the open/closed state of the front covers mounted on the support members 93 based on the detection result of the rotation detection unit 95. As a detailed arrangement, in the breast imaging apparatus 110, the front cover 9 has a structure (opening/closing structure) divided into left and right portions. The divided front covers 9 are mounted on the support members 93. The front covers 9 are provided with a circular opening 91 to make a breast of an object as a body part to be imaged enter. Each support member 93 is rotatably supported with respect to the rotation axis of the rotation support unit 94 with a hinge structure as the rotation center. When the support member 93 rotates with respect to the rotation axis of the rotation support unit 94 as the rotation center, the front cover 9 mounted on the support member 93 is opened/closed. FIG. 8A shows a state (closed state) in which the front covers 9 are closed. The rotation detection unit 95 that detects the rotation of the rotation axis of the rotation support unit is arranged on the rotation axis of the rotation support unit 94. The detection result of the rotation detection unit 95 is input to the control unit 200. The rotation detection unit 95 can detect the rotation angle of the rotation axis of the rotation support unit 94 corresponding to opening/closing of the left and right support members 93. The rotation detection unit 95 outputs the information of the detected rotation angle to the control unit 200. Detailed control of the control unit 200 will be described later with reference to the control block diagram of FIG. 11 and FIG. 12. Note that as the arrangement of the rotation detection unit 95, for example, a detection mechanism such as a rotary encoder or a resolver can be used.

In the breast imaging apparatus 110 according to this embodiment, a cover detection unit 152 that detects the mounted state of a cover that separates the object from the radiation imaging unit and a handle detection unit 154 that detects the grip of a handle by the object can be mounted on the support member 93 in place of the fixed frame 5 described in the first embodiment. A contact detection unit 156 that detects the presence/absence of the object can be arranged in front of the front covers 9 having the opening/closing structure.

In the breast imaging apparatus 110 according to this embodiment, the cover detection unit 152 can detect, as the mounted state of a cover, the presence/absence of mounting of the cover or the position of the cover. The cover detection unit 152 includes a first detection unit that detects the presence/absence of mounting of the cover as the mounted state of the cover, and a second detection unit that detects the position of the cover. An arrangement that switches between mammogram imaging and CBCT imaging by the control unit 200 based on the mounted state of the cover (the detection result of the presence/absence of mounting of the cover) has been described in the first embodiment. In this embodiment, an arrangement that switches between mammogram imaging and CBCT imaging by the control unit 200 based on the detection results of the positions of the covers that separate the object from the radiation imaging unit as the mounted states of the covers will be described.

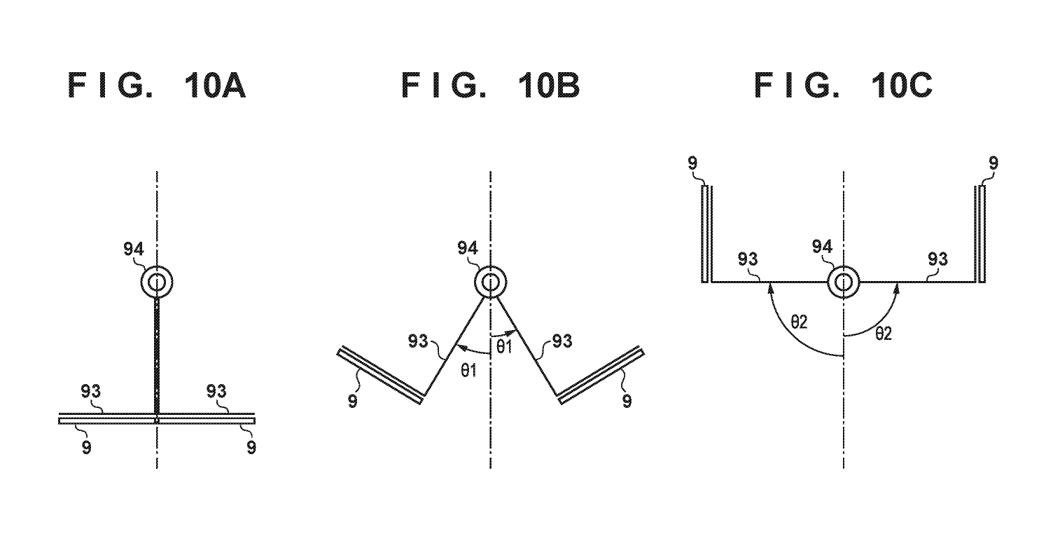

The cover detection unit 152 is arranged in correspondence with each of the left and right front covers 9. The cover detection units 152 can detect the positions of the covers as the mounted states of the covers based on, for example, detection signals corresponding to the relative positions of the left and right cover detection units 152. Each cover detection unit 152 can detect, as the position of the cover, a position corresponding to a closed state (FIG. 10A) in which the front cover 9 is closed, a position corresponding to an open state (FIG. 10C) in which the front cover 9 is opened, and a position corresponding to an intermediate state (FIG. 10B) between the closed state and the open state. For example, the output level of a detection signal corresponding to the state (closed state) in which the front cover 9 is closed is defined as a closed state signal level, and the output level of a detection signal corresponding to the state (open state) in which the front cover 9 is opened is defined as an open state signal level. In addition, the output level of a detection signal corresponding to the intermediate state of the front cover 9 is opened is defined as an intermediate state signal level. The relative relationship of the signal levels is expressed as closed state signal level>intermediate state signal level>open state signal level.

For example, if the output of the detection signal of the cover detection unit 152 has the closed state signal level, the cover detection unit 152 detects, as the mounted state of the cover, that the cover is located at the position corresponding to the closed state. If the output of the detection signal of the cover detection unit 152 has the open state signal level, the cover detection unit 152 detects, as the mounted state of the cover, that the cover is located at the position corresponding to the open state. If the output of the detection signal of the cover detection unit 152 has the intermediate state signal level, the cover detection unit 152 detects, as the mounted state of the cover, that the cover is located at the position corresponding to the intermediate state. The control unit 200 may determine the position of the cover corresponding to the open/closed state of the front cover mounted on the support member 93 based on the detection result of the cover detection unit 152. The control unit 200 can switch between mammogram imaging and CT imaging based on the position of the cover as the mounted state of the cover.

FIGS. 10A to 10C are views showing states of the front covers 9 having the opening/closing structure. FIGS. 10A to 10C are views schematically showing the rotation support unit 94, the support members 93, and the front covers 9 viewed from above the breast imaging apparatus 110. FIG. 10A shows the state (closed state) in which the front covers 9 are closed. In the closed state in which the front covers 9 are closed, the rotation angle (θ) of the rotation axis of the rotation support unit 94 is 0° (θ=0°) with respect to a rotation standard indicated by an alternate long and short dashed line passing through the center of the rotation axis. The rotation detection unit 95 outputs the rotation angle θ=0° of the rotation axis of the rotation support unit 94 to the control unit 200 as a detection result.

FIG. 10B shows a state (intermediate state) in which the front covers 9 begins opening from a closed state (closed state) or the front covers 9 begins closing from an opened state (open state). In the intermediate state, the rotation detection unit 95 outputs a rotation angle θ1 (0<θ1<θ2) of the rotation axis of the rotation support unit 94 to the control unit 200 as a detection result. For example, if the rotation angle changes from θ=0° to θ1, the control unit 200 determines that the front covers 9 change from the closed state to the intermediate state.

FIG. 10C shows the state (open state) in which the front covers 9 are opened. In the open state in which the front covers 9 are opened, the rotation angle (θ) of the rotation axis of the rotation support unit 94 is θ=θ2. Here, θ2 is a preset angle and serves as a threshold angle used to determine whether the covers (front covers) are in the open state. θ2 can arbitrarily be set. For example, θ2 can be set to an angle closed to 90°. The rotation detection unit 95 outputs the rotation angle θ2 of the rotation axis of the rotation support unit 94 to the control unit 200 as a detection result. For example, if the rotation angle changes from θ1 to θ2, the control unit 200 determines that the front covers 9 change from the intermediate state to the open state.

The control unit 200 can determine the open/closed state of the front covers 9 based on the detection result of the rotation detection unit 95. For example, if the rotation angle of the rotation axis is 0° with respect to the rotation standard, the control unit determines that the covers are in the closed state (FIG. 10A: when the detection result of the rotation detection unit 95 is 0° (θ=0°)).

If the detection result of the rotation detection unit 95 is 0<θ1<θ2, the control unit 200 determines that the front covers 9 are in the intermediate state between the closed state and the open state (FIG. 10B). If the rotation angle is equal to or larger than the angle set to the rotation standard, the control unit 200 determines that the covers are in open state. That is, if the detection result of the rotation detection unit 95 is θ≧θ2, the control unit 200 determines that the front covers 9 are in the open state (FIG. 10C).

In the control of CT imaging, the control unit 200 can control the operations of the radiation generation unit 10 and the rotation unit 57 based on the detection results of the cover detection units 152, the detection result of the rotation detection unit 95, and the detection result of the object detection unit. Detailed control of the control unit 200 will be described later with reference to the control block diagram of FIG. 11 and FIG. 12.

FIGS. 9A and 9B are views for explaining the schematic arrangement of the breast imaging apparatus 110 according to the second embodiment when performing mammogram imaging. FIG. 9A shows the state (open state) in which the front covers 9 are opened. FIG. 9B shows a state in which the apparatus with the opened front covers 9 is viewed from a side. The same reference numerals as in the first embodiment denote the same components such as the radiation generation unit 10, the radiation detection unit 20, and the housing unit 4, and a description thereof will be omitted.

A pressing plate support unit 31 and a pressing plate 3 are removable from the rotation unit 57, that is, a radiation imaging unit 2. If the pressing plate support unit 31 and the pressing plate 3 are kept placed on the rotation unit 57, they hinder an imaging technician from doing imaging when performing CBCT imaging. Hence, at the time of CBCT imaging, the pressing plate support unit 31 is removed from the rotation unit 57 together with the pressing plate 3, as shown in FIGS. 8A and 8B. At the time of mammogram imaging, the pressing plate support unit 31 is held by the rotation unit 57 together with the pressing plate 3, as shown in FIGS. 9A and 9B.

In the control of mammogram imaging, the control unit 200 can control the operations of the radiation generation unit 10 and the rotation unit 57 based on the detection results of the cover detection units 152, the detection result of the rotation detection unit 95, and the detection result of a pressing plate detection unit 158. Detailed control of the control unit 200 will be described later with reference to the control block diagram of FIG. 11 and FIG. 12.

FIG. 11 is a block diagram showing for explaining processing of the control unit 200 of the breast imaging apparatus 110. The breast imaging apparatus 110 includes the cover detection unit 152, the handle detection unit 154, the contact detection unit 156, the pressing plate detection unit 158, and the rotation detection unit 95 as the components of the detection unit. Each detection unit is connected to the control unit 200, and the detection result of each detection unit is input to the control unit 200. The control unit 200 controls the overall operation of the breast imaging apparatus 110. More specifically, the control unit 200 can control the operation of the radiation imaging unit 2 (the radiation generation unit 10, the radiation detection unit 20, the rotation unit 57, and the like) of the breast imaging apparatus 110 based on the detection result of each detection unit. A selection unit 300 that selects an imaging type is connected to the control unit 200. The control unit 200 can switch between mammogram imaging and CT imaging based on the selected imaging type. The selection unit 300 can select the imaging type based on an input by the imaging technician via a user interface (display unit) (not shown).

FIG. 12 is a view for explaining processing of the control unit 200 of the breast imaging apparatus 110. When detection information of an ON state representing the mounted state of the front cover 9 is input from each cover detection unit 152 to the control unit 200, the control unit 200 determines that the front covers 9 are mounted (front cover mounting: present) and controls the imaging type of the radiation imaging unit 2 to CT imaging (CBCT imaging).

On the other hand, when detection information of an OFF state representing the unmounted state of the front cover 9 is input from each cover detection unit 152 to the control unit 200, the control unit 200 determines that the front cover 9 is unmounted (front cover mounting: absent) and controls the imaging type of the radiation imaging unit 2 to mammogram imaging. That is, the control unit 200 performs imaging control to switch between mammogram imaging and CT imaging based on the detection results of the cover detection units.

In the control of CT imaging (CBCT imaging), the control unit 200 determines the open/closed state of the front covers 9 based on the detection result of the rotation detection unit 95. If the detection result of the rotation detection unit 95 is θ=θ2, as shown in FIG. 10C, the control unit 200 determines that the front covers 9 are in the open state. When the front covers 9 are in the open state, the control unit 200 controls the rotation unit 57 of the radiation imaging unit 2 to an unrotatable state and controls the radiation generation unit 10 to a radiation irradiation disable state. In this case, the imaging operation of the radiation imaging unit 2 is disabled by the control of the control unit 200. In CBCT imaging, since the rotation time in the 360° section is about 5 sec, the control is done to prevent the object or the imaging technician from coming into contact with the rotation unit 57.

In the control of CT imaging (CBCT imaging), if the detection result of the rotation detection unit 95 is θ=0°, as shown in FIG. 10A, the control unit 200 determines that the front covers 9 are in the closed state. If detection information of an OFF state representing the ungripped state of a handle by the object is input from the handle detection unit 154 in the case in which the front covers 9 are in the closed state, the control unit 200 controls the rotation unit 57 of the radiation imaging unit 2 to a rotatable state but controls the radiation generation unit 10 to a radiation irradiation disable state. In this case, the imaging operation of the radiation imaging unit 2 is disabled by the control of the control unit 200. Even if the detection information of the OFF state is input from the handle detection unit 154 to the control unit 200, the rotation unit 57 is controlled to the rotatable state because the rotary frame 6 needs to be rotated when returning to the initial position of CBCT imaging before shifting from mammogram imaging to CBCT imaging. Regardless of whether the detection result of the handle detection unit 154 indicates the ON state or OFF state, the control unit 200 controls the rotation unit 57 to the rotatable state. Hence, in the operation of the rotary frame 6 when returning to the initial position of CBCT imaging, the operability for the imaging technician can be improved.

In the control of CT imaging (CBCT imaging), if detection information of an ON state representing the gripped state of a handle by the object is input from the handle detection unit 154 to the control unit 200 in the case in which the front covers 9 are in the closed state, the control unit 200 controls the rotation unit 57 of the radiation imaging unit 2 to a rotatable state and controls the radiation generation unit 10 to a radiation irradiation enable state. In this case, the control unit 200 controls to enable imaging by the radiation imaging unit 2. That is, even in the closed state of the front covers 9, if the object does not grip the handle of the handle detection unit 154 (OFF state), CT imaging (CBCT imaging) cannot be started.

When the object releases the handle of the handle detection unit 154 during CT imaging (CBCT imaging), the handle detection unit 154 changes from the ON state to the OFF state. Detection information of the OFF state is then input from the handle detection unit 154 to the control unit 200. Based on the detection information of the handle detection unit 154 representing the OFF state, the control unit 200 controls the radiation generation unit 10 and stops radiation irradiation from the radiation generation unit 10. That is, the control unit 200 controls the radiation generation unit 10 of the radiation imaging unit 2 to a radiation irradiation disable state, and the imaging operation of the radiation imaging unit 2 is disabled by the control of the control unit 200.

Note that FIG. 12 shows the handle detection unit as an example of the detection result of the object detection unit that detects the state of the object. However, the control unit 200 can also control the operation of the radiation imaging unit 2 using the detection result of not the handle detection unit but the contact detection unit 156 that detects the presence/absence of the object.

In the control of mammogram imaging, the control unit 200 determines the open/closed state of the front covers 9 based on the detection result of the rotation detection unit 95. If the detection result of the rotation detection unit 95 is θ=θ2, as shown in FIG. 10C, the control unit 200 determines that the front covers 9 are in the open state. When detection information of an OFF state representing the unpressed state of the breast by the pressing plate 3 is input from the pressing plate detection unit 158 in the case in which the front covers 9 are in the open state, the control unit 200 controls the rotation unit 57 of the radiation imaging unit 2 to a rotatable state but controls the radiation generation unit 10 to a radiation irradiation disable state. In this case, the imaging operation of the radiation imaging unit 2 is disabled by the control of the control unit 200.

In the control of mammogram imaging, when detection information of an ON state representing the pressed state of the breast by the pressing plate 3 is input from the pressing plate detection unit 158 to the control unit 200 in the case in which the front covers 9 are in the open state, the control unit 200 controls the rotation unit 57 of the radiation imaging unit 2 to an unrotatable state (limits the rotation to a minute angle range (for example, 1° to 2°)), and controls the radiation generation unit 10 to a radiation irradiation enable state. In this case, the imaging operation of the radiation imaging unit 2 is enabled by the control of the control unit 200. The rotation is limited to the minute angle range (for example, 1° to 2°) because the angle of MLO may finely be adjusted in the breast pressed state. To cope with such fine adjustment, even if the pressing plate detection unit 158 outputs the detection information of the ON state representing the pressed state, the control unit 200 can also control the radiation generation unit 10 to the radiation irradiation enable state in a state in which the rotation angle of the rotation unit 57 (rotary frame 6) is limited to a minute angle (for example, 1° to 2°).

Note that when controlling CT imaging (CBCT imaging) or mammogram imaging, if the state of the front covers 9 is the intermediate state, for example, the control unit 200 can also perform display control to display a warning message for the imaging technician via a user interface (display unit) (not shown) to avoid contact between the rotation unit 57 and the support members 93.

As described above, according to the arrangements of the embodiments, it is possible to provide a breast imaging apparatus capable of performing both mammogram imaging and CT imaging while ensuring safety for an object when performing imaging.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2015-131838, filed Jun. 30, 2015, which is hereby incorporated by reference herein in its entirety.

What is claimed is:

1. A breast imaging apparatus including a radiation imaging unit capable of performing mammogram imaging and CT imaging, comprising:
a cover detection unit configured to detect a state of a cover that separates an object from the radiation imaging unit; and
a control unit configured to switch between the mammogram imaging and the CT imaging based on the state.

2. The apparatus according to claim 1, wherein the cover detection unit detects one of presence/absence of mounting of the cover and a position of the cover as the state of the cover.

3. The apparatus according to claim 1, wherein based on a detection result of the cover detection unit, the control unit
controls the CT imaging if the cover is mounted, and
controls the mammogram imaging if the cover is not mounted.

4. The apparatus according to claim 1, further comprising a selection unit configured to select an imaging type,
wherein the control unit switches between the mammogram imaging and the CT imaging based on the selected imaging type.

5. The apparatus according to claim 1, wherein the radiation imaging unit comprises:
a radiation generation unit configured to generate radiation;
a radiation detection unit configured to detect the radiation; and
a rotation unit capable of rotating the radiation generation unit and the radiation detection unit in a state in which the radiation generation unit and the radiation detection unit face each other.

6. The apparatus according to claim 5, further comprising:
an object detection unit configured to detect an object in front of the cover; and
a pressing plate detection unit configured to detect that a body part of the object to be imaged is pressed by a pressing plate mounted on the radiation imaging unit.

7. The apparatus according to claim 6, wherein in control of the CT imaging, the control unit controls operations of the radiation generation unit and the rotation unit based on a detection result of the cover detection unit and a detection result of the object detection unit.

8. The apparatus according to claim 6, wherein in control of the mammogram imaging, the control unit controls operations of the radiation generation unit and the rotation unit based on a detection result of the cover detection unit and a detection result of the pressing plate detection unit.

9. The apparatus according to claim 6, further comprising:
a rotation support unit arranged in a housing unit of the breast imaging apparatus;
a support member rotatably supported with respect to a rotation axis of the rotation support unit as a rotation center and configured to be able to mount the cover; and
a rotation detection unit configured to detect a rotation of the rotation axis,
wherein the control unit determines an open/closed state of the cover mounted on the support member based on a detection result of the rotation detection unit.

10. The apparatus according to claim 9, wherein the control unit
determines that the cover is in a closed state if a rotation angle of the rotation axis is 0° with respect to a rotation standard, and
determines that the cover is in an open state if the rotation angle is not less than an angle set to the rotation standard.

11. The apparatus according to claim 9, wherein in the control of the CT imaging, the control unit controls the operations of the radiation generation unit and the rotation unit based on the detection result of the cover detection unit, the detection result of the rotation detection unit, and the detection result of the object detection unit.

12. The apparatus according to claim 9, wherein in the control of the mammogram imaging, the control unit controls the operations of the radiation generation unit and the rotation unit based on the detection result of the cover detection unit, the detection result of the rotation detection unit, and the detection result of the pressing plate detection unit.

13. The apparatus according to claim 1, wherein the cover is provided with an opening to insert a body part of the object to be imaged.

14. The apparatus according to claim 1, wherein the cover is configured to be removable from the breast imaging apparatus.

15. A method of controlling of a breast imaging apparatus including a radiation imaging unit capable of performing mammogram imaging and CT imaging, comprising:
detecting a state of a cover that separates an object from the radiation imaging unit; and
switching between the mammogram imaging and the CT imaging based on the state.

16. A computer-readable storage medium storing a program that causes a computer to execute each step of a method of controlling of a breast imaging apparatus including a radiation imaging unit capable of performing mammogram imaging and CT imaging, the method of controlling the breast imaging apparatus comprising:
detecting a state of a cover that separates an object from the radiation imaging unit; and
switching between the mammogram imaging and the CT imaging based on the state.
